(19) United States
(12) Patent Application Publication
(10) Pub. No.: US 2012/0238777 A1
(43) Pub. Date: Sep. 20, 2012

(54) METHOD AND DEVICE FOR CONCENTRATING MATERIAL SOLUTIONS

(75) Inventors: Michael Harasek, Wien (AT); Saeed Gul, Wien (AT)

(73) Assignee: TECHNISCHE UNIVERSITAT WIEN, WIEN (AT)

(21) Appl. No.: 13/394,859

(22) PCT Filed: Sep. 8, 2010
(86) PCT No.: PCT/AT2010/000320

§ 371 Date: Jun. 1, 2012

(30) Foreign Application Priority Data
Sep. 11, 2009 (AT) A 1446/2009

(51) Int. Cl. B01D 61/02; C07C 51/47; B01D 61/10; C07C 227/40; C13B 20/16; B01D 61/14

(52) U.S. Cl. 562/554; 127/55; 210/650; 210/652; 210/175; 210/253; 562/580

(57) ABSTRACT

A method is provided for concentrating a material solution using at least two consecutive membrane separating stages, each stage for separating the material solution into a retentate and a permeate. The cut-off of the membranes used in the separating stages is higher than that of the membrane used in the previous stage. The retentate of each separating stage, with the exception of the last stage, is fed to the following separating stage as a feed, and the permeate from at least one separating stage is fed back to a preceding separating stage and introduced into the feed thereof. The fed-back permeate has a concentration and viscosity that substantially corresponds to the concentration and viscosity of the preceding separating stage into which the permeate is introduced.

### CROSS-REFERENCE TO RELATED APPLICATIONS

This application is a Section 371 of International Application No. PCT/AT2010/000320, filed Sep. 8, 2010, which was published in the German language on Mar. 17, 2011, under International Publication No. WO 2011/029110 A1 and the disclosure of which is incorporated herein by reference.

### BACKGROUND OF THE INVENTION

The present invention relates to a method and a device for concentrating material solutions by pressure-powered membrane procedures.

In numerous large-scale production methods, the desired products are obtained in the form of solutions more or less strongly diluted, from which the respective useful material has to be separated by complex separation methods. Representative examples thereof are the sugar, fruit juice, pharmaceutical, and chemical industries.

In sugar production, for example, energy is responsible for the second highest expenses of all operating costs because, at the end of refining, large amounts of water have to be removed from the obtained sucrose solution—typically by evaporation. Since the sugar beet at the beginning of the process consists of approximately 75% water, and further approximately 20% water are added in the course of the procedure, water accounts for about 95% of the sugar solution. Evaporating this water content requires enormous amounts of energy, because of the high heat of evaporation of water.

Consequently, there have been attempts to separate part of that water before the evaporation process. From U.S. Pat. No. 3,617,550 it is known to concentrate solutions by passing two reverse-osmosis separating stages through membranes of different permeabilities, wherein optionally a permeate from the second stage is recycled into the feed of the first stage. Exemplary solutions mentioned are a diluted sugar solution and fruit juices, and the inventors calculate the required operating pressures for each of the two separating stages from the retention rate of the membrane separation. In U.S. Pat. No. 5,096,590 a similar system having numerous separating stages (up to twelve) is disclosed, wherein each last permeate is led back into the feed of the first separating stage.

While this procedure has resulted in a noticeable concentration effect, the procedure still has disadvantages with regard to energy demand as well as pressure and temperature ratios within the devices, partly because the maximum operating pressure possible is limited by the selection of the membranes. It is thus an object of the present invention to improve these methods according to the prior art.

### BRIEF SUMMARY OF THE INVENTION

In a first aspect, this object is achieved by providing a method wherein a material solution is concentrated by using at least two consecutive membrane separating stages, each for separating the material solution into a retentate and a permeate, the cut-off of the membranes used in the separating stages being higher than that of the membrane used in the respective preceding stage, the retentate of each separating stage, with the exception of the last stage, being supplied to the subsequent separating stage as a feed, and the permeate from at least one separating stage being returned to a preceding separating stage and introduced into the feed thereof, wherein the method is characterized by the returned permeate having a concentration or viscosity that substantially corresponds to the concentration or viscosity of the feed of the preceding separation stage into which it is introduced.

The viscosities of the process streams depend not only on their temperature, but mainly also on the concentration of the substances dissolved therein, especially, of course, of the useful material to be separated. By regulating the concentration or viscosity as described above, mixing the returned permeate with the feed, into which it is introduced, is facilitated and accelerated, which results in more uniform operating conditions and thus in an improved throughput of the procedure, without the requirement of using a higher operating pressure. There are fewer control-related fluctuations in permeate streams of preceding stages with which returned permeates are mixed. Further essential advantages are that by adjusting the properties, the overall energy expenditure of the treatment procedure is lower (due to higher pressure in the pumps), and the overall method may be arranged in a simpler way. If the concentration of a returned permeate is too high, much larger membrane surfaces are required in the preceding stage due to higher osmotic pressures; on the other hand, if the concentration of a returned permeate is too low, the procedure is uneconomical because the feed of the preceding stage is unnecessarily diluted, which may lead to increased pumping requirements and also larger membrane surfaces or other hydraulic conditions in the membrane separation module.

Herein, the phrase that a concentration “substantially corresponds” to another one refers to a difference between the two concentrations of not more than 3 wt %. If viscosities are the decisive values, a viscosity that “substantially corresponds” to another one refers to a maximum difference between the viscosities of 10% to 15% because—especially when using aqueous sugar solutions—the viscosity changes, in the case of low concentrations (i.e. approximately 15 to 20% solutions), by approximately 3.5% if the concentration is increased by 1 wt %, and in the case of higher concentrations (i.e. approximately 35 to 40% solutions) by approximately 5%. Of course, in cases in which the temperature of the processed material solution does not change, controlling the concentration goes hand in hand with controlling the viscosity.

Especially in cases without temperature changes, preferred embodiments of the invention contemplate a difference between the concentrations of the returned permeate and the feed of the preceding membrane separating stage of not more than 3 wt %, more preferably not more than 2 wt %, particularly not more than 1 wt %, so that the advantages described above are expressed to a particularly high extent.

The number of separating stages contemplated in the inventive method is not particularly limited. If a material solution is subjected to three or more separating stages, the permeate of each separating stage, starting with the second one, is preferably introduced into the feed of the immediately preceding separating stage, which further increases the efficiency of the method.

In especially preferred embodiments, only the first separating stage is charged with an external feed-side operating pressure, and in each separating stage a membrane is used having a permeability such that the difference between the osmotic pressures of the retentate and the permeate is lower than the respective feed-side operating pressure. Thus, no further pumps or similar pressure devices are required after the first separating stage, because the initially set operating pressure is sufficient to conduct the material solution through all separating stages.

Preferably, in the first separating stage, a membrane is used which has a cut-off that is smaller than the size of the material molecules to be separated, so that practically no useful material is lost in the first separating stage. In a preferred separating method, in the first membrane separating stage, either reverse osmosis or nanofiltration is conducted using a cut-off that is lower than the molecular mass of the useful material, so that material solutions may be processed that essentially consist of only a solvent and the useful material. In the second and each further optional membrane separating stage, however, preferably nanofiltration or ultrafiltration is conducted, so that part of the useful material may pass through the membrane and the appropriate concentration or viscosity is achieved in the respective permeate stream.

The material solution to be separated in the inventive method is not particularly limited. As described above, sugar solutions or fruit juice may be used, as well as solutions of lactic acid or a salt thereof, amino acid solutions or other aqueous, non-aqueous or mixed aqueous/non-aqueous solutions as are common, for example, in chemical industry, pharmaceutical chemistry, biotechnology, etc.

Especially preferred, a sugar solution is processed in the inventive method, particularly a hot, refined sugar solution. By using a hot solution, which in sugar production from sugar beets or sugar cane commonly has a temperature of 80° C., pressure loss in the membrane modules decreases strongly because viscosity is much lower at higher temperatures, without the requirement of additionally heating the solution. Thus, much less energy is needed for pumping the solution through the system and also in a subsequent evaporation unit, because the retentate from the last membrane separating stage already has a higher temperature.

Of course, there are also other material solutions in the form of hot solutions in the respective production method, and these solution are preferably also fed into the inventive method in a hot state. Here, it might be necessary to provide temperature-resistant membranes in the separating stages.

In a second aspect, the present invention relates to a device for implementing a preferred embodiment of the inventive method, namely a device for concentrating a material solution comprising the following: a) at least two serially connected membrane separation modules having a feed conduit, a retentate conduit and a permeate conduit, wherein the retentate conduit of each separation module, with the exception of the last one, simultaneously constitutes the feed conduit of the subsequent separation module or leads into it, wherein at least one permeate conduit leads into the feed conduit of a preceding separation module, and wherein the cut-off value of the membranes provided in the separation modules is higher than that of the membrane provided in the respective preceding separation module; b) at least one pressure device for pressurizing at least one separation module; and c) optionally one or more heating and/or cooling elements; wherein the device is characterized in that at least three membrane separation modules are provided and that the permeate conduits of the separation modules, starting with the second one, lead into the feed conduits of the respective preceding separation modules.

By returning all permeates into the feeds of preceding separating stages, the separating efficiency of the separating method conducted therein is strongly increased compared to the prior art. Preferably, the permeate conduit of each separation module, starting with the second one, leads, in the flow direction of the material solution, into the separation module positioned directly upstream, so that the concentrations are more easily adjustable, as has been described with regard to the method according to the first aspect of the invention.

Since permeate streams are basically unpressurized, preferred embodiments of the inventive device provide further pressure devices for feed streams consisting of the retentate of a preceding and the permeate of a subsequent separating stage. Particularly, for each separation module having a permeate conduit leading into the feed conduit thereof, a pressure device is provided to pressurize the separation module. The additional pressure devices guarantee a high throughput of the inventive device and a uniform flow therein without the requirement of extremely high power input into the first pressure device.

In further preferred embodiments, the first separation module comprises a membrane having a cut-off that is smaller than the size of the material molecules to be separated, so that substantially no valuable material is separated in the first separation module. Preferably, the first separation module is a reverse-osmosis module, because it allows especially small cut-off values, or a nanofiltration module having a cut-off that is lower than the molecular mass of the valuable material, so that material solutions may be processed that essentially consist of only a solvent and a valuable material. The second and optionally each further separation module is/are preferably (a) nanofiltration module(s) or (an) ultrafiltration module(s) having a higher cut-off value in order to allow part of the valuable material to pass the membrane and thus adjust the appropriate concentration or viscosity in the respective permeate stream.

In order to be able to also process very hot material streams, preferably at least one membrane of a separation module is a temperature-resistant membrane. If the temperature of the material solution is to be kept high within the entire device, more preferably each membrane used therein is temperature-resistant. In further preferred embodiments, at least one membrane of at least one separation module is a ceramic membrane, because they are characterized by high thermal and chemical stability as well as very good purification possibilities.

Furthermore, preferably at least one membrane of at least one separation module is a membrane having especially high permeability, i.e. a high-flux membrane. This either increases the throughput of the device, or smaller membrane surfaces may be used at the same throughput. In addition, the membranes may have lower retention or higher cut-off values.

Those skilled in the art will understand that the inventive device may comprise not only one or more optional heating and/or cooling elements for cooling or heating individual separation modules and/or corresponding feed and discharge conduits, but in addition comprises or may comprise further standard components, such as additional pumps, pressure-relief or back-pressure valves, bypass conduits, pressure gauges, temperature sensors, or the like.

Furthermore, it will be obvious to the skilled artisan that the components, especially the membranes within the individual separating stages, are not particularly limited as long as the requirements defined above and in the attached claims are met, especially the specifications regarding the cut-off. For each of the separating stages, standard membranes from any manufacturer may be used, such as Novasep, GE Osmonics, Hydranautics, Dow Water & Process Solutions, Toray, Koch Membrane Systems, etc.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a flow chart of the inventive method in its simplest embodiment within a device according to the prior art; and

FIG. 2 is a flow chart of a preferred embodiment of the inventive method within a device according to the present invention.

### DETAILED DESCRIPTION OF THE INVENTION

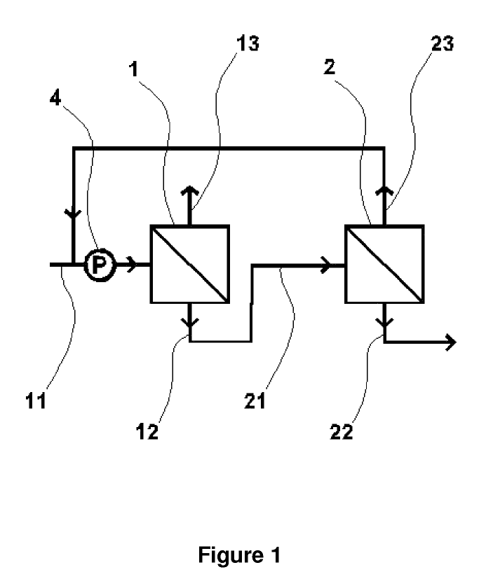

FIG. 1 shows a standard device for implementing a two-stage membrane separating method as known, for example, from U.S. Pat. No. 3,617,550. A material solution, e.g. a sugar solution, is introduced via a feed conduit 11 into a first membrane module 1 that is pressurized by a pressure device 4, usually a pump.

Within separation module 1, the solution is separated into a retentate and a permeate, wherein the former is discharged via a retentate conduit 12 and the latter via a permeate conduit 13. The permeate obtained from 13, which usually has a very low material concentration, is thus usually discarded, while the retentate obtained from 12 is introduced via a feed conduit 21 into a second separation module 2 to subject it to a second separating stage. The retentate obtained therefrom is discharged via a retentate conduit 22 and constitutes the desired concentrated material solution, while the second permeate, which usually has a higher material concentration than the first one, is recycled into the first separation module via a permeate conduit 23.

During this recycling, the following problems occur in the prior art: There are control-related fluctuations of the permeate stream in the first separating stage so that the pump 4 has to provide relatively high pressure in order to balance the fluctuations. If the concentration of the recycled permeate is too high, a significantly larger membrane surface is required in the first separating stage because of the higher osmotic pressure. A relatively low concentration of the returned permeate, however, is uneconomical because in this case, the returned permeate unnecessarily dilutes the feed of the first stage, which again requires more pumping power and larger membrane surfaces.

According to the present invention, such a separating method is thus optimized by adjusting the concentration and/or viscosity of the returned permeate, such that it essentially corresponds to the concentration of the feed solution with which the permeate is mixed. This means that the difference between the concentrations amounts to not more than 3 wt % or the difference between the viscosities amounts to not more than 15%, preferably not more than 2 wt % or not more than 10%, even more preferably not more than 1 wt % or not more than 5%.

Such an adjustment of the concentration or viscosity according to the invention is obtained by an appropriate design of the overall system, wherein special focus is given to the separating membranes, the respective pressure ratios and optionally the temperatures of the individual streams. Since any material solutions may be treated according to the invention, which may have very different osmotic pressures, each separating system requires an empirical determination of the best parameters for the respective case, since—apart from the above preferred embodiments of the inventive method and the inventive device—general instructions for the layout of such separating systems are not possible. However, specific information on individual defined systems can be found in the following exemplary embodiments.

As is also shown in FIG. 1, in a preferred embodiment of the inventive method only the first separating stage 1 receives operating pressure from the feed side by a pump 4, and each separating stage uses a membrane having a permeability such that the difference in the osmotic pressures of the respective retentate and permeate is lower than the feed-side operating pressure. Thus, no further pumps or the like are necessary, which strongly reduces the energy requirements of the method.

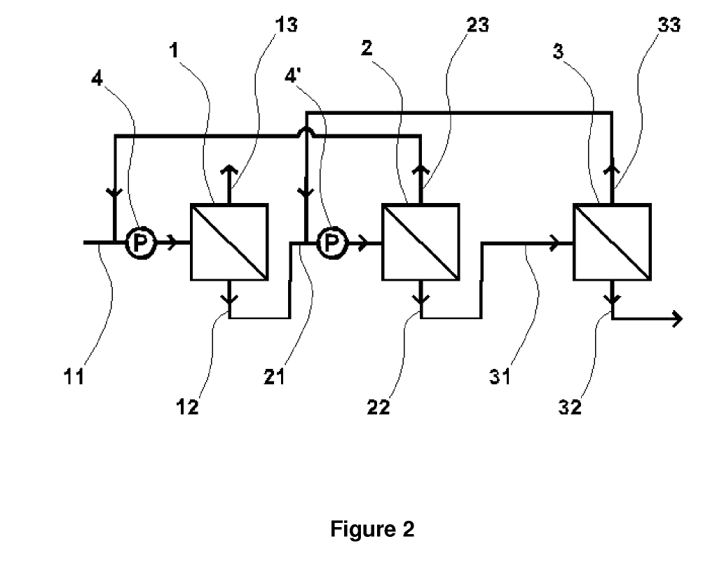

However, the present invention does not only relate to two-stage separating methods. Rather, the number of separating stages is not limited, and in preferred embodiments of the inventive method, at least three separating stages are used, as is shown in FIG. 2. Consequently, this figure shows a flow chart of a preferred embodiment of the method of the invention and schematically a device according to a second aspect of the invention for implementing this preferred embodiment of the inventive method.

Compared to FIG. 1, the device of FIG. 2 is extended by a third separation module 3, into which the retentate obtained from the retentate conduit 22 of the second separation module is introduced via a corresponding feed conduit 31. This feed is again separated, in which case the solution discharged via retentate conduit 32 constitutes the desired concentrated material solution.

In this three-stage method, two permeates are returned, each into the respectively preceding separating stage, as is preferred according to the present invention, which facilitates the adjustment of the required maximum concentration difference between the permeate and the feed into which it is introduced. Thus, in FIG. 2, the permeate of the second separating stage is discharged via the permeate conduit 23 and introduced into the feed conduit 11 of the first stage, while the permeate of the third separating stage is discharged via the permeate conduit 33 and introduced into the feed conduit 21 of the second stage.

Since permeate streams are always unpressurized, it might be necessary to provide for a second pump 4′ for the mixed feed into the second stage so that the pump 4 does not have to provide excessively high pressure, but, nevertheless, an inventive device having a high throughput and at the same time providing a uniform flow of the material solution therein is obtained.

The skilled artisan will be easily able to extend the inventive method and the inventive device to comprise four or more separating stages, preferably comprising a separate pump in front of each separating stage due to the above reasons.

By conducting an at least three-stage method, the concentration of a concentrated material solution may be further increased, which in the case of a refined sugar solution as starting feed of the method, saves even more energy in the subsequent evaporation process.

Below, the invention is described in more detail by three specific exemplary embodiments using sugar solutions as material solutions, wherein it is obvious that the examples are only given for illustrative but not limiting purposes.

### EXAMPLES

### Examples 1 to 3

The following exemplary embodiments simulate two- and three-stage separating methods by use of a refined sugar solution, i.e. an aqueous solution of sucrose at a temperature of 40° C., with the assumption that the temperature will not change significantly within the system, which in practice is achievable by high throughputs of the individual separation modules as well as, optionally, additional heating of the modules and/or conduits.

The two-stage Example 1 is an exemplary embodiment of the inventive method only, while Examples 2 and 3 illustrate the method as well as the device of the invention.

For the simulations, the following membranes were used:

Filmtec NF270 by Dow Water & Process Solutions, a plastic nanofiltration membrane based on piperazine/polyamide having a cut-off of 270 Da;

TFC-SR3 by Koch Membrane Systems, a plastic nanofiltration membrane based on polyamide having a cut-off of 200-300 Da;

SelRo MPF-36 by Koch Membrane Systems, a plastic nanofiltration membrane having a cut-off of 1000 Da, the material of which is not further specified by the manufacturer; and

Kerasep BW 1.0 kD and 1.2 kD by Novasep, nanofiltration membranes based on ceramic monoliths having cut-offs of 1000 or 1200 Da.

The following Table 1 shows the individual stages of the membrane combinations used in the examples together with the corresponding operating pressures (in bars), percentage retentions of sucrose, specific flows (kg/(m²·hr)), feed concentrations, retentate concentrations, and mean permeate concentrations of the individual stages (in wt % sucrose).

TABLE 1

| | | | Operating | | | Feed | End | Permeate |
|---|---|---|---|---|---|---|---|---|
| | | | pressure | Retention | Spec. flow | conc. | conc. | conc. |
| Example | Stage | Membrane | [bar] | [%] | [kg/(m² · hr)] | [wt %] | [wt %] | [wt %] |
| 1 | 1 | FilmTec NF270 | 33 | 98.8 | 28.5 | 15.0 | 20.0 | 0.2 |
| | 2 | SelRo MPF-36 | 32 | 47.9 | 17.9 | 20.0 | 40.0 | 15.0 |
| 2 | 1 | FilmTec NF270 | 33 | 99.0 | 32.1 | 12.0 | 18.0 | 0.2 |
| | 2 | SelRo MPF-36 | 32 | 48.5 | 21.9 | 18.0 | 29.0 | 12.0 |
| | 3 | Kerasep BW 1.0 | 28 | 46.9 | 30.5 | 29.0 | 40.0 | 18.2 |
| 3 | 1 | TFC-SR3 | 33 | 99.7 | 20.2 | 15.0 | 25.0 | 0.1 |
| | 2 | SelRo MPF-36 | 32 | 47.9 | 17.6 | 25.0 | 31.4 | 15.0 |
| | 3 | Kerasep BW 1.2 | 31 | 35.9 | 26.5 | 31.4 | 45.0 | 24.5 |

The above table shows that with the inventive adjustment of the returned permeate stream to essentially the same concentration as that of the feed into which it is introduced, a material solution may be efficiently concentrated. All concentration differences between permeates and feeds mixed with each other were below 1 wt %, as is especially preferred according to the invention.

A direct comparison of Examples 1 and 2 shows that by adding a third separating stage to the system, the concentrating effect was not increased, but the flow through the device was increased. On the other hand, Example 3 shows a much stronger concentrating effect of the material solution than Example 1, so that the subsequent evaporation process required less energy than according to the prior art.

Thus, the examples clearly show that by appropriately selecting the separating membranes and the other essential parameters of a two- or more-stage membrane separating method, the concentrations of the recycled permeates can very accurately be adjusted to those of the feed solutions of preceding stages, which results in substantial improvements of the overall separating method.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1.-21. (canceled)

22. A method for concentrating a material solution containing a useful material to be concentrated, the method comprising:
providing at least two consecutive separating stages, wherein each of the separating stages separates the material solution into a retentate and a permeate,
using a separation membrane in each of the separating stages which has a cut-off that is higher than a cut-off of the separation membrane used in a respective preceding separating stage,
feeding the retentate of each separating stage, except a last separating stage, to a following separating stage as a feed, and
feeding the permeate from at least one separating stage back to a preceding separating stage and introducing this permeate into a feed of the preceding separating stage,
wherein the permeate fed back to the feed of the preceding separating stage has a concentration and viscosity that substantially correspond to a concentration and viscosity of the feed of the preceding separating stage into which the fed back permeate is introduced, such that the concentrations differ by not more than 3 wt % or the viscosities differ by not more than 15%, respectively.

23. The method according to claim 22, wherein the concentration difference is not more than 2 wt %.

24. The method according to claim 23, wherein the concentration difference is not more than 1 wt %.

25. The method according to claim 22, wherein the material solution is subjected to at least three separating stages and the permeate of each separating stage, starting with a second separating stage, is reintroduced into the feed of an immediately preceding separating stage.

26. The method according to claim 22, wherein only a first separating stage is charged with an external feed-side operating pressure, and in each separating stage the separation membrane has a permeability such that a difference between osmotic pressures of the retentate and the permeate is lower than a respective feed-side operating pressure.

27. The method according to claim 22, wherein, in a first separating stage, the separation membrane has a cut-off smaller than a size of molecules of the useful material to be concentrated.

28. The method according to claim 22, wherein, in a first separating stage, either reverse osmosis or nanofiltration is conducted using a membrane having a cut-off lower than a molecular mass of the useful material to be concentrated.

29. The method according to claim 22, wherein, in a second and any further separating stage, nanofiltration or ultrafiltration is conducted using a cut-off lying within or above a range of a molecular mass of the useful material to be concentrated.

30. The method according to claim 22, wherein the material solution comprises a sugar solution.

31. The method according to claim 30, wherein the sugar solution is a hot refined sugar solution.

32. The method according to claim 22, wherein the material solution comprises a solution of lactic acid or a salt of lactic acid.

33. The method according to claim 22, wherein the material solution comprises an amino acid solution.

34. A device for concentrating a material solution containing a useful material to be concentrated, the device comprising the following:
a) at least three serially connected membrane separation modules, each of the separation modules having a feed conduit, a retentate conduit, and a permeate conduit, wherein the retentate conduit of each separation module, except a last of the separation modules in a flow direction of the material solution, simultaneously constitutes or leads into the feed conduit of a subsequent separation module,
wherein each permeate conduit, starting with a second one of the separation modules in the flow direction of the material solution, leads into the feed conduit of a preceding separation module, and
wherein a cut-off of a membrane provided in a separation module is higher than a cut-off of the membrane provided in a respectively preceding separation module;
b) at least one pressure device for pressurizing at least one of the separation modules; and
c) optionally one or more heating and/or cooling elements.

35. The device according to claim 34, wherein the permeate conduit of each separation module, starting with the second one, leads into the feed conduit of the separation module positioned directly upstream of the flow direction.

36. The device according to claim 34, wherein a first separation module comprises a membrane having a cut-off smaller than a size of molecules of the useful material to be concentrated.

37. The device according to claim 34, wherein a first separation module is a reverse-osmosis module or a nanofiltration module comprises a membrane having a cut-off lower than a molecular mass of the useful material to be concentrated.

38. The device according to claim 34, wherein the second one and any further separation modules are selected from nanofiltration and ultrafiltration modules having a cut-off within or above a range of a molecular mass of the useful material to be concentrated.

39. The device according to claim 34, wherein at least one membrane of at least one separation module is a temperature-resistant membrane.

40. The device according to claim 34, wherein at least one membrane of at least one separation module is a ceramic membrane.

41. The device according to claim 34, wherein at least one membrane of at least one separation module is a high-flux membrane.

42. The device according to claim 34, wherein for each of the separation modules having a permeate conduit leading into the feed conduit of a preceding separation membrane a pressure device is provided to pressurize the feed to the preceding separation membrane.
